(19) United States
(12) Patent Application Publication
(10) Pub. No.: US 2018/0017541 A1
(43) Pub. Date: Jan. 18, 2018

(54) SYSTEMS AND METHODS TO DETECT AND MEASURE MATERIALS IN OIL

(71) Applicant: William Brian Kinard, Atherton, CA (US)

(72) Inventor: William Brian Kinard, Atherton, CA (US)

(21) Appl. No.: 15/645,397

(22) Filed: Jul. 10, 2017

Related U.S. Application Data
(60) Provisional application No. 62/362,467, filed on Jul. 14, 2016.

(51) Int. Cl. G01N 33/28; G01N 21/76; G01N 21/64

(52) U.S. Cl. CPC G01N 33/2888; G01N 33/2858; G01N 33/2864; G01N 21/643; G01N 33/2835; G01N 21/76; G01N 2021/6439

(57) ABSTRACT

An ion concentration measurement system is provided. The system includes an oil mixture comprising an oil potentially containing at least one ion and at least one photo-responsive chemical that changes optical properties in the presence of at least one ion in the oil mixture, photo-detector circuitry configured to receive an optical property from the oil mixture, electronic conversion circuitry configured to convert the optical property into a value representing an ion concentration in the oil mixture, and electronic transmission circuitry configured to transmit a signal or data indicating the value. A method of measuring concentrations of at least one ion in an oil mixture is provided, including receiving at least one optical property from at least one photo-responsive chemical, converting the at least one optical property into a value representing an ion concentration in the oil mixture, and transmitting a signal or data comprising the value.

The present application claims the benefit of U.S. Provisional Patent Application Ser. No. 62/362,467, filed Jul. 14, 2016, inventor William Brian Kinard, entitled “Systems and Methods to Detect and Measure Materials in Oil,” the entirety of which is incorporated herein by reference.

### BACKGROUND OF THE INVENTION

### Field of the Invention

The present invention generally relates to systems and methods for detecting, measuring and/or monitoring materials in oil, and more specifically, systems and methods for measuring or monitoring the condition of lubricating oil in a mechanical system for the presence of metal or other materials in the oil for the purposes of measuring and monitoring quality of the lubricating oil, wear on the mechanical system, or contamination in the mechanical system.

### Description of the Related Art

Concentrations of materials in the oil of a mechanical system is an indication and measure of wear and other conditions in the mechanical system. These issues in turn may indicate or predict problems with the lubricating properties of the oil, contamination in the oil or mechanical system, and/or problems with the mechanical system itself.

Predictive maintenance of mechanical systems is more ecological, efficient, and cost-effective than routine maintenance. Routine maintenance of mechanical systems entails maintaining the mechanical system within specified operating parameters by regularly timed maintenance intervals, such as regular oil changes and/or regular inspection and exchange of wear components. In contrast to routine maintenance, predictive maintenance of mechanical systems measures the operating parameters of the mechanical system, including wear, and predicts when maintenance and/or failure of the mechanical system may or will occur. Prior designs do not provide a simple, robust, inexpensive, in situ or portable, real-time system or method to predict maintenance or wear in a mechanical system.

Three general inefficiencies exist in lubricated mechanical systems—first, lubricating too frequently, causing extra operating costs and harm to the environment, second, lubricating too infrequently, thereby causing engine wear and subsequent drops in energy efficiency and/or power, and third, at the risk of lubricating too infrequently and causing machine failure, over-engineering the mechanical systems, resulting in unnecessary costs.

Lubricating too frequently does not cause direct machine wear or failure, but the costs and ecological damage are significant. Over 600 million gallons of lubricating oil are used in the United States every year. In the United States, the average car owner changes oil less than every 5,000 miles; while in Europe, the average car owner changes oil more than every 10,000 miles. If 10,000 miles is the optimum average oil-change interval, then car owners in the United States waste over 300 million gallons of oil at a cost of $1.5 billion, not including labor, filter costs etc. In addition, approximately 30% of motor oil is not collected for waste or recycling, resulting in an environmental hazard that is exacerbated by over-lubrication.

Lubricating too infrequently has costly effects on machine wear, lifetime and operating costs. Overextended oil drain intervals increase engine wear by an average of more than 20%, with a corresponding reduction in horsepower and fuel efficiency.

Lastly, because under-lubrication can cause premature engine failure, most mechanical systems are over-engineered. Basic life (or L₁₀) is the life that 90% of a sufficiently large group of apparently identical mechanical bearings can be expected to reach or exceed. The median or average life, sometimes called mean time between failure (MTBF), is up to five to ten times L₁₀. The specification life is generally a requisite L₁₀ basic rating life and reflects a manufacturer's requirement based on experience with similar applications. A bearing's failure can have catastrophic effects and costs, much greater than the cost of the bearing itself. Further, unpredicted maintenance causes unplanned downtime, resulting in significant collateral costs. For example, unplanned maintenance of a mining or manufacturing mechanical system causes unplanned labor and output costs (i.e. fixed costs remain while revenue decreases). Earlier and more accurate prediction of a bearing's failure will allow engineers to design mechanical systems with cheaper bearings for the same application thereby reducing the costs of mechanical systems.

There is therefore a need for a durable, low-cost, on-board oil quality and/or mechanical system quality sensor capable of: (i) identifying optimal mechanical system oil drain intervals, including mechanical system cycle, mechanical system condition, original oil quality, and oil property measurements in real-time; (ii) identifying or predicting mechanical system failures, including excessive wear; (iii) communicating warnings and/or signals and recommended action to operators and mechanics; and/or (iv) logging fault codes for abusive operating and maintenance procedures.

In sum, it would be beneficial to provide systems and methods for detecting and measuring the existence of materials in oil that overcomes issues with previously known techniques, reducing waste and increasing lifespan of mechanical systems as compared with prior designs.

### SUMMARY OF THE INVENTION

According to one aspect of the present design, there is provided an ion concentration measurement system, comprising an oil mixture comprising an oil potentially containing at least one ion and at least one photo-responsive chemical that changes optical properties in the presence of at least one ion in the oil mixture, photo-detector circuitry configured to receive an optical property from the oil mixture, electronic conversion circuitry configured to convert the optical property into a value representing an ion concentration in the oil mixture, and electronic transmission circuitry configured to transmit a signal or data indicating the value.

According to another aspect of the present design, there is provided a method of measuring concentrations of at least one ion in an oil mixture comprising oil potentially containing at least one ion and at least one photo-responsive chemical that changes at least one optical property in the presence of at least one ion in oil. The method comprises receiving the at least one optical property from the at least one photo-responsive chemical, converting the at least one optical property into a value representing an ion concentration in the oil mixture, and transmitting a signal or data comprising the value.

According to a further embodiment of the present design, there is provided a method of measuring condition of a mechanical system comprising combining data in at least one additional mechanical system capable of measuring concentration of materials in oil with other parameters of the mechanical system, wherein the other parameters comprise first mechanical system operating parameters since last oil change, original oil quality, and first mechanical system current and historic load and operating conditions, implementing statistical learning algorithms configured to learn typical oil or machine degradation patterns for a specific mechanical system type to determine mechanical system-specific oil drain histories, and employing the mechanical system-specific oil drain histories to optimize subsequent oil drain intervals for the first mechanical system.

These and other advantages of the present invention will become apparent to those skilled in the art from the following detailed description of the invention and the accompanying drawings.

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1 is a simplified energy diagram showing typical fluorescence mechanisms and steps;

FIG. 2 is a functional, cutaway block diagram showing photo-responsive chemicals in oil in a mechanical system;

FIG. 3 is a flow chart showing the process of measuring the concentration of materials in oil in a mechanical system for purposes of measuring oil condition, contamination or wear of the mechanical system;

FIG. 4 is a graph of the light-intensity spectrum of one photo-responsive chemical in oil;

FIG. 5 is a functional, cutaway block diagram showing photo-responsive chemicals attached to a sight glass in a mechanical system; and

FIG. 6 is a flow chart showing the process of measuring the concentration of materials in oil in a mechanical system for purposes of measuring oil condition, contamination or wear of the mechanical system.

The exemplification set out herein illustrates particular embodiments, and such exemplification is not intended to be construed as limiting in any manner.

### DETAILED DESCRIPTION

The following is provided to facilitate an understanding of some of the innovative features unique to the disclosed embodiments and is not intended to be a full description. A full appreciation of the various aspects of the embodiments disclosed herein can be gained by taking the entire descriptions and claims contained herein. The particular values and configurations discussed in the non-limiting examples can be varied and are cited merely to illustrate at least some embodiments and are not intended to limit the scope thereof.

In the following description, reference is made to “some embodiments.” Note that “some embodiments” describes a subset of all of the possible embodiments, but does not always specify the same subset of embodiments. Further, this does not limit the permutations or combinations of all or parts of embodiments in the present invention.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure. The recitation of numerical ranges by endpoints includes all numbers within that range (e.g. 2 to 4 includes 2, 2.3, 3, 3.60, and 4).

As used herein and in the appended claims, the singular forms “a”, “an”, and “the” include plural referents unless the content clearly dictates otherwise. As used herein and in the appended claims, the term “or” is generally employed in its sense including “and/or” unless the content clearly dictates otherwise.

As used herein and in the appended claims, use of the word “including” means also “including without limitation” unless the content clearly dictates otherwise.

As used herein and in the appended claims, the word “ion” or “ionic” includes any configuration of atoms or poly-atomic molecules that have or had positive or negative net electronic charges.

As used herein and in the appended claims, the word “material” includes any configuration of atoms or poly-atomic molecules, including ions. As non-limiting examples, materials include metals, metal ions, carbon, sulfur, contaminants and/or water.

As used herein and in the appended claims, the word “derivative” when referencing a specific material or chemical includes (i) materials or chemicals that are derived from that specific chemical, (ii) materials or chemicals used to derive that specific material or chemical, or (ii) materials or chemicals resulting from bonding, building or attaching atoms, molecules and/or chemicals to that specific chemical. As a non-limiting example, chemicals such as 8-hydroxyquinoline thioamide, 8-hydroxyquinoline acetamide and 8-hydroxyquinoline-5-sulfonic acid are derivatives of 8-hydroxyquinoline.

As used herein and in the appended claims, the term “mechanical system” includes any configuration of mechanical parts that periodically or continuously undergo linear, rotational, or other motion, internally and/or externally, and includes additional parts such as housings, blocks, casings, fluids, valves, and other parts that facilitate the mechanical motion. As non-limiting examples, “mechanical system” includes engines, motors, gearboxes, transmissions, hydraulic systems, pumps, mixers and braking systems; and use of the word “engine,” “motor” etc. herein is meant to include all mechanical systems, unless the context clearly indicates otherwise.

As used herein and in the appended claims, the term “measurement system” includes systems and methods of some embodiments that are not attached to the mechanical system but rather are handheld and/or portable embodiments that permit the measurement system to measure ion concentrations of materials in mechanical systems.

While the plural is used in many descriptions herein, it is to be understood that the terminology is intended broadly, and use of the plural indicates in most instances one or more of the recited elements. For example, “Optical properties and/or color energy spectrums hν₂ 540 are dependent on several parameters, including excitation energies hν₁ 538, photo-responsive chemicals 520, ions 518 in oil 514 . . . ” is intended to indicate one or more optical properties and/or one or more color energy spectrums hν₂ 540 are dependent on several parameters, including one or more excitation energies hν₁ 538, one or more photo-responsive chemicals 520, one or more ions 518 in oil 514. This is the case for use of the plural throughout this document, and the use of the singular is not intended to specifically exclude the plural.

The present design generally includes systems and methods for detecting, measuring and/or monitoring materials in oil. The present design includes, without limitation, systems and methods for measuring or monitoring the condition of lubricating oil in a mechanical system for the presence of metal or other materials in the oil for the purposes of measuring and monitoring (i) the quality of the lubricating oil, (ii) wear on the mechanical system or (iii) contamination in the mechanical system.

In one aspect, photo-responsive chemicals are introduced in the oil, and the photo-responsive chemicals change color, excitation spectra, or fluorescence intensity, polarity, lifetime or wavelength depending on the concentrations of materials in the oil. In some embodiments, these changes in colors or excitation spectra, fluorescence intensities, polarities, lifetimes or wavelengths are detected and measured by an electronic circuit such as a photo-spectrometer, a colorimeter or color filters coupled with photo-detectors. In some embodiments, these electronic signals are correlated and/or converted to the concentrations of materials in the oil and are transmitted to an operator or computer for further analysis. The above-mentioned systems and methods may serve as an inexpensive, robust, in situ monitor and predictor of problems with the mechanical system, either in the lubrication oil and/or in the mechanical system itself.

In another aspect, the mechanical system is assembled in a manner that allows oil to be exposed to the photo-responsive chemical(s). The photo-responsive chemical(s) may be introduced in the oil itself. The photo-responsive chemical(s) may be bound to a mechanical structure, such as a sight glass or dipstick, that is in contact with the oil. The photo-responsive chemical(s) may be bound to mechanically stable, solid chemicals, such as silica gel, which is then exposed to the oil directly or is bonded to a mechanical structure, such as a sight glass or dipstick, which is in contact with the oil.

Control logic may be implemented by a programmed microprocessor determining and/or computing the concentrations of material(s) in oil by measuring values or changes in color or fluorescence intensity, polarity, lifetime or wavelength over a measured time interval. An increase in the concentrations of materials in oil over time indicates the presence of contamination, lubrication deterioration and/or mechanical system problems.

Embodiments of the present design provide an improved in situ oil quality or machine quality sensor and methods of using the same. Some embodiments provide an in situ oil sensor capable of identifying oil drain intervals and detecting mechanical system malfunction or incipient failure, while some embodiments provide an in situ oil sensor capable of identifying contamination in the oil and/or mechanical system.

Other embodiments provide an on-board, in situ oil sensor in an oil lubricated mechanical system, including a powered vehicle, capable of identifying optimal engine oil drain intervals based upon (i) the concentrations of materials in oil and (ii) other measurable parameters such as engine cycle and condition, original oil quality, and engine load conditions. Certain embodiments provide an on-board, in situ oil sensor capable of identifying mechanical system failures based upon (i) the concentrations of metals in oil and/or the concentrations of other materials in oil and (ii) other measurable parameters, including engine cycle and condition, original oil quality, and engine load conditions.

Still other embodiments provide an on-board, in situ oil analyzer capable of communicating warnings and recommended actions to operators and mechanics and logging fault codes indicating abusive operating procedures and/or conditions. Other embodiments combine (i) the systems or methods described above in mechanical systems capable of measuring the concentrations of materials in oil with (ii) other mechanical system parameters, including mechanical system cycle and condition, original oil quality, and mechanical system load conditions and (iii) statistical learning algorithms, resulting in a “smart sensor system” capable of learning typical oil degradation patterns for a specific mechanical system type and using these mechanical system-specific oil drain histories to optimize subsequent oil drain intervals not only for the specific mechanical system being measured but also other similarly configured and operated mechanical systems.

Further, some embodiments combine one or more of the systems or methods described above capable of measuring the concentrations of one or more materials in oil with an off-board measurement and monitoring computer capable of moving or being transported among one or more mechanical systems to measure the concentrations of materials in oil among one or more mechanical systems, and such embodiments include handheld sensors, sensors attached to integrated manufacturing systems and the like, known herein as “measurement systems.”

Some embodiments detect mechanical system malfunctions or incipient failures by analyzing the oil for concentrations of one or more materials in oil and other measurable parameters, including mechanical system cycle and condition, original oil quality, and mechanical system load conditions.

Embodiments disclosed herein provide a durable, low-cost, on-board oil quality and/or mechanical system quality sensor capable of: (i) identifying optimal mechanical system oil drain intervals, including mechanical system cycle, mechanical system condition, original oil quality, and oil property measurements in real-time; (ii) identifying or predicting mechanical system failures, including excessive wear; (iii) communicating warnings and/or signals and recommended action to operators and mechanics; and/or (iv) logging fault codes for abusive operating and maintenance procedures.

Methods are also provided for monitoring the quality of oils in mechanical systems. The methods include continually monitoring the optical properties of photo-responsive chemicals in the presence of the oil, that interact with materials, to detect changes in the efficacy of the oil, storing values representative of the concentrations of materials in a memory, performing trend analyses of the stored values to characterize variations of the stored values, and estimating a recommended oil change interval based on at least one of the trend analyses. Performing trend analyses may include examining variations in oil parameters since a previous oil change, over total engine operating hours, or over a predetermined portion of the total engine operating hours. In some embodiments, trend analyses include comparing a current mechanical system's oil measurement and rate of change to previously stored values to determine the type of oil added to the mechanical system. In some embodiments, the method includes monitoring changes in engine oil viscosity, material concentrations in oil, and other engine and oil parameters, and determining a recommended oil change interval based on changes in the viscosity and changes in the concentrations of materials in the oil.

In some embodiments, methods include determining whether oil contamination may be present and alerting and/or signaling an operator in response thereto. In still other embodiments, methods determine whether oil has been added to the lubrication system by detecting a decrease in concentrations of materials in the oil, coupled possibly with a change in oil level. In some embodiments, when oil addition is detected, stored calibration data are modified to adjust future calculations of recommended oil change intervals. Methods also include automatic detection of an oil change by detecting significant changes in at least one measured oil parameter.

Thus some embodiments provide simple, low-cost, in situ or portable systems and methods to measure wear of a mechanical system and provide information to predict maintenance or failure of the mechanical system. Other embodiments introduce photo-responsive chemicals to the oil in a mechanical system, and the photo-responsive chemicals interact with materials in the oil and change the photo-responsive chemical's fluorescence, color, polarity or other optical properties based on the concentrations of materials in the oil.

As used in this specification and the appended claims, “optical properties” include without limitation, fluorescence intensity, fluorescence wavelength, fluorescence anisotropy, absorption intensity, absorption wavelength, absorption anisotropy, excitation spectra, color, chemiluminescence of various forms, phosphorescence of various forms, ratios of the above-mentioned, and any changes in the above-mentioned over time or changing environmental conditions. Measuring these optical properties provides direct measurement of materials in the oil and thereby detects, measures and/or monitors wear or contamination in the mechanical system. Some embodiments of the present invention provide data about mechanical-system wear and predictions about lubrication, contamination, maintenance or failure of a mechanical system.

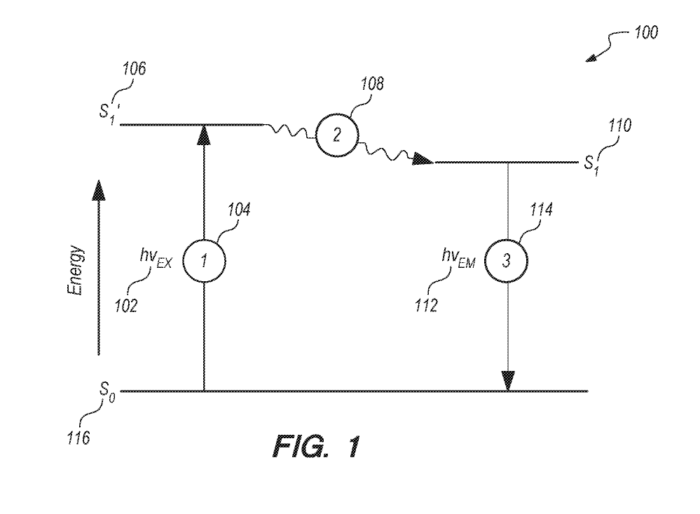

In general, fluorescence is the result of a three-stage process that occurs in certain atoms or molecules, including fluorophores (also known as fluorescent dyes). The three-stage process responsible for fluorescence is described in the energy diagram of FIG. 1.

As used in this specification and the appended claims, “photo-responsive chemicals” includes any chemical that changes its fluorescence intensities, fluorescence wavelengths, polarity, fluorescence lifetime, absorption color, reflection color, transmission color or other optical properties in the presence of and/or by interacting with materials, and includes (i) fluorophores, (ii) chromophores, (iii) oil itself, and (iv) chemicals that interact with metal, other materials, ions, oil and/or other chemicals to create chemiluminescence. Further, photo-responsive chemicals include a plurality of molecules (one or more of which are chelators that bind to ions and one or more of which are fluorophores); and the molecules interact with each other, including resonant energy transfer, to change the combined molecules' optical properties based on chelation to ions. As a non-limiting example, a chelator is a compound that bonds to an ion.

As used in this specification and the appended claims, the word “chromophore” includes any molecule or groups of molecules that change their transmission, absorbance or reflection color in response to interacting with ions.

The representation of FIG. 1 includes three steps. Step 1 is Excitation. In Step 1 104, external sources, including light-emitting diodes, incandescent lamps or lasers, supplies a photon 102 of energy hν_{EX} (where ν_{EX} is the frequency of the photon and h is Planck's constant). Photon 102 is absorbed by the fluorophore, creating an excited electronic singlet state (S₁′) 106. This process distinguishes fluorescence from chemiluminescence, in which the excited state is created by a chemical reaction.

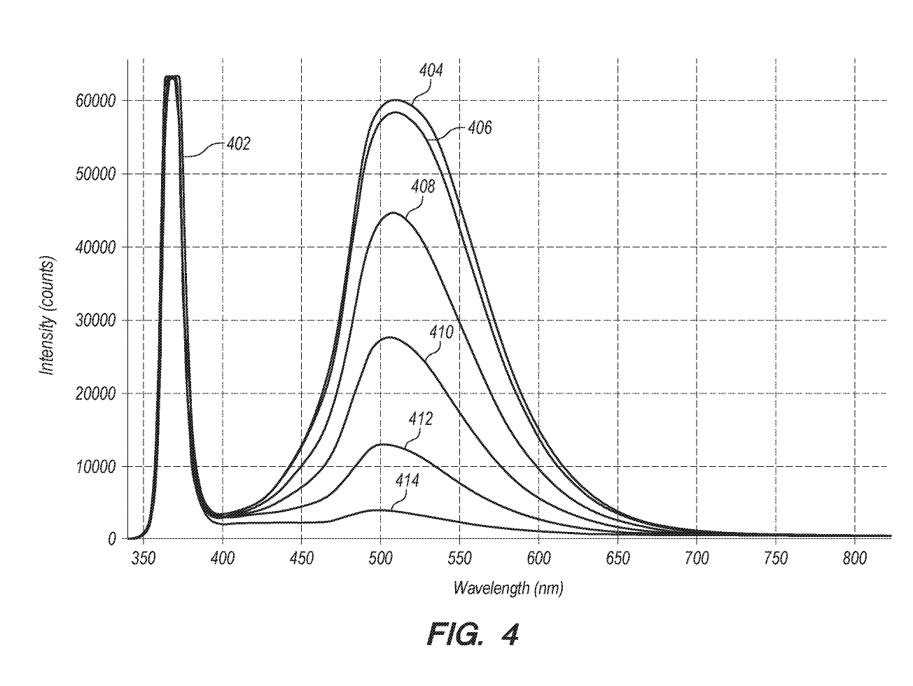

Step 2 is Excited-State Lifetime and Interactions. In Step 2 108, the excited state exists for a finite time (typically 1-10 nanoseconds, but can be as long as milliseconds for some materials including lanthanides). During this time, the fluorophore undergoes conformational changes and is typically subject to a multitude of possible interactions with its molecular environment 108. These processes have two notable consequences. First, the energy of S₁′ 106 is partially dissipated, yielding a relaxed singlet excited state (S₁) 110 from which fluorescence emission originates. Second, not all the molecules initially excited by absorption (Step 1) return to the ground state (S₀) 116 by fluorescence emission. Other processes (including collisional quenching, fluorescence resonance energy transfer (FRET) and intersystem crossing) may also depopulate S₁ 110, sometimes without significant fluorescence emission. The fluorescence quantum yield, representing the ratio of the number of fluorescence photons emitted (Step 3) to the number of photons absorbed (Step 1), is a measure of the relative extent to which these processes occur. This can be noteworthy in the present design because the fluorescence frequencies, fluorescence lifetimes, fluorescence polarities and/or fluorescence intensities of one or more wavelengths change based on the molecular interaction between the fluorophores and the materials in their surrounding environment in certain circumstances. For example, 8-hydroxyquinoline fluoresces in oil, and 8-hydrozyquinoline binds or chelates to ions (including iron ions (Fe³⁺), aluminum ions (Al³⁺) and other ions) in the oil, changing its optical properties. FIG. 4 shows one example of the change in optical properties of a photo-responsive chemical in the presence of iron ions.

Step 3 of FIG. 1 represents Fluorescence Emission. In Step 3 114, a photon of energy hν_{EM} 112 is emitted, returning the fluorophore to its ground state S₀ 116. Due to energy dissipation during the excited-state lifetime, the energy of this photon (hν_{EM} 112) is lower, and therefore of longer wavelength, than the excitation photon hν_{EX} 102. The difference in energy or wavelength represented by (hν_{EX} 102-hν_{EM} 112) is called the Stokes shift. The Stokes shift is fundamental to the sensitivity of fluorescence techniques, allowing emission photons to be detected against a low-energy background, isolated from excitation photons. In contrast, absorption spectrophotometry requires measurement of transmitted light relative to high incident light levels at the same wavelength, decreasing the sensitivity and resolution of absorption spectrophotometry relative to fluorescence photometry.

In some embodiments, the entire fluorescence process 100 is cyclical. Unless the fluorophore is irreversibly destroyed in the excited state (known as photobleaching), the same fluorophore can be repeatedly excited and detected. As a single fluorophore can generate many thousands or millions of detectable photons, the result is that the present design is robust and exhibits high sensitivity. For polyatomic molecules, the discrete electronic transitions represented by hν_{EX} 102 and hν_{EM} 112 in FIG. 1 are replaced by broader energy spectra called the fluorescence excitation spectrum and fluorescence emission spectrum, respectively. The bandwidths of these spectra can be of particular importance for certain embodiments, such as those wherein two or more different fluorophores are simultaneously detected. With few exceptions, the fluorescence excitation spectrum of a single fluorophore species in dilute solution is identical or nearly identical to its absorption spectrum. Under the same conditions, the fluorescence emission spectrum is independent of the excitation wavelength due to the partial dissipation of excitation energy during the excited-state lifetime, as illustrated in FIG. 1 108. The emission intensity is proportional to the amplitude of the fluorescence excitation spectrum at the excitation wavelength.

In some embodiments, the absorbance of excitation energy or energies (known as excitation spectra) change with increased concentrations of ions in oil; and this change is detected, measured and converted to concentrations of materials in oil.

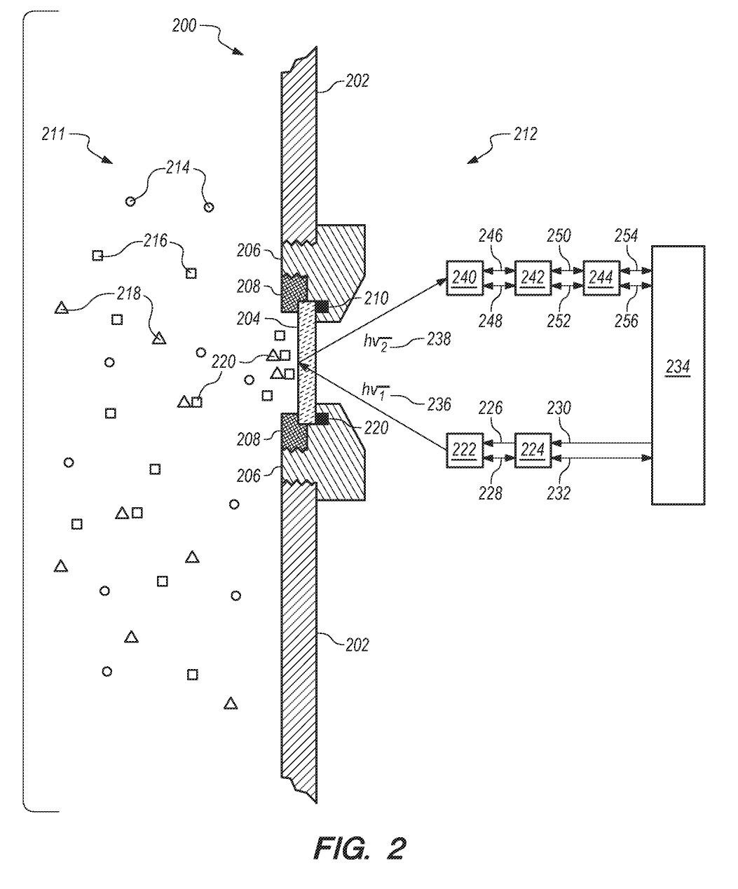

The block diagram of FIG. 2 shows measurement of concentrations of ions in oil in a mechanical system 200, including a side cutaway view of the mechanical enclosure 202 and sight glass fixture (204-210, and 220). Most oil-lubricated mechanical systems 200 are encased in a mechanical enclosure 202 that holds oil 211. The oil 211 serves four principal purposes for the mechanical system 200—(i) lubrication of the moving parts in the mechanical system 200, (ii) inhibiting rust and corrosion, (iii) distributing and/or dissipating heat in the mechanical system 200 and/or (iv) distributing and/or dissipating heat through the filter, removing contaminants, including soot (principally carbon and sulfur) and larger objects from further causing problems in the mechanical system 200. In some mechanical systems, the oil 211 may be recirculated though the mechanical system 200 via an oil pump and circulation channels. Alternately, the oil 211 may be agitated and mixed by the motion of the parts of the mechanical system 200 itself.

Some embodiments include a sight glass fixture (204-210, and 220). In some embodiments, the transparent sight glass 204 permits excitation energies hν₁ 236 from excitation sources 222 to enter the mechanical system 200 and interact with the oil 211 and/or photo-responsive chemicals 218 and/or permits electromagnetic energy hν₂ 238 to exit the oil 211 and/or photo-responsive chemicals 218 to be detected by photo-detector circuits 240. The sight glass fixture (204-210, and 220) can be custom built or obtained from commercial sources. Further, sight glass fixtures can be in many shapes and forms, including circular or cylindrical. In some embodiments, the sight glass fixture (204-210, and 229) is circular and attached to the mechanical enclosure 202, which can include the block, oil pan, oil conduit, casing or oil filter cover for the mechanical system. One example of a circular sight glass fixture is W.E. Anderson Sight Window (P/N SFI-550-3/4), a division of Dwyer Instruments, Inc. (www.dwyer-inst.com). The sight glass fixture (204-210, 220) may be cylindrical and attached to the oil recirculating system of the mechanical system 200. One example of a cylindrical sight glass fixture is Dixon Valve & Coupling B54BMP-R150 In-Line Sight Glass.

In some embodiments, the circular sight glass fixture (204-210, and 220) comprises four basic parts: (i) the sight glass 204, made of various materials including fused glass or plastic depending on the operating parameters (including temperature and pressure) of the oil 211 in the mechanical system 200; (ii) sight glass fixture casing 206 upon which the sight glass 204 rests; (iii) circular gasket 210 and 220, preventing oil 211, metal or other materials 214, ions 216 and photo-responsive chemicals 218 from escaping the mechanical system 200 and entering the exterior environment 212, including air; and (iv) a circular flange 208 that screws into the sight glass fixture casing 206 and presses against the sight glass 204 and circular gasket 210 and 220, preventing movement of the sight glass 204 and preventing oil 211, metal or other materials 214, ions 216 and photo-responsive chemicals 218 from escaping the mechanical system 200 and entering the exterior environment 212.

In some embodiments, photo-responsive chemicals 218 are introduced into the oil 211, including periodically or immediately after each oil change. One example of a photo-responsive chemical is 8-hydroxyquinoline (C₉H₇NO) (CAS 148-24-3) from Alfa Aesar (P/N 41272). 8-hydroxyquinoline is a chelator, which means that these molecules attach to ions and possibly other materials. Further, 8-hydroxyquinoline is a fluorophore and fluoresces in oil when it is excited at ultraviolet energy. In some embodiments, when 8-hydroxyquinoline binds with increasing concentrations of ions in oil, the intensity of fluorescence increasingly quenches or increases and/or one or more of the wavelengths of fluorescence shift. As one non-limiting example, 8-hydroxyquinoline fluoresces brightly at about 510 nm in fresh oil, and the intensity of fluorescence and the fluorescence peak wavelength decrease for increased concentrations of iron in oil. FIG. 4 illustrates embodiments showing the quenching and shifting of fluorescence of iron ions in oil. In some embodiments, 8-hydroxyquinoline dissolves or is miscible in oil and fluoresces at an excitation wavelength of about 365 nanometers, meaning the light source 222 can be comprised of lower-cost, readily-available 365 nanometer light emitting diodes. In some situations, as little as 100 parts per million (ppm) of 8-hydroxyquinoline in oil 211 is sufficient to chelate and measure ion concentrations of the range of certain embodiments of the present design, making 8-hydroxyquinoline an inexpensive photo-responsive chemical for some uses. In some applications, 8-hydroxyquinoline has been demonstrated to be robust and show little degradation in its optical properties over a range of temperatures up to 350 degrees Fahrenheit in oil. Further, 8-hydroxyquinoline has not been shown to adversely affect the operation of the oil 211 or mechanical system 200. In some embodiments, other photo-responsive chemicals are used to measure the concentrations of ions in oil, including the following photo-responsive chemicals: calcein and/or calcein derivatives, cyanine and/or cyanine derivatives, rhodamine and/or rhodamine derivatives, Texas Red and Texas Red derivatives, and/or 8-hydroxyquinoline and/or 8-hydroxyquinoline derivatives.

Atoms and/or poly-atomic molecules may be added to or bound to the photo-responsive chemicals in order to enhance their photo-responsive properties, including selectivity to certain materials or ions, sensitivity, fluorescence quantum yield, fluorescence intensity multiple, quenching fraction, wavelength of peaks of the fluorescence spectra, and shifting of wavelengths of peaks of the fluorescence spectra. These enhanced photo-responsive chemicals are known as derivatives of the above described photo-responsive chemicals. As a non-limiting example, molecules may chelate to ions in oil, changing the molecule's chemical or electrical properties. This change is transferred (via resonance energy transfer or otherwise) to other molecule(s) in which one or more optical properties change. In the aggregate, this junction of these different types of molecules (chelators and fluorophores) serves as an effective sensor. The photo-responsive chemicals may change their color in response to interactions with ions or other materials in oil.

In some embodiments, independently or on conjunction with fluorescence, color-changing photo-responsive chemicals determine the concentration of ions or other materials in oil by measuring the gradual change in color of the photo-responsive chemicals in response to gradual changes in the concentration of ions or other materials in the oil. Photo-responsive chemicals may be attached to certain solid chemicals, including silica which has very small pores and a very high ratio of surface area to volume. In such a situation, certain ions access the photo-responsive chemicals, increasing the selectivity of the photo-responsive chemical to certain ions in the oil.

In some situations, oil 211 contains very little metal or other materials 214 immediately after each oil change, although fresh oil has been found to contain iron and/or aluminum up to 3 ppm each. Although oil 211 is hydrophobic, some water and other chemicals exist in oil. Up to one gallon of water may be produced for every gallon of fuel consumed in some gasoline and diesel internal combustion engines; and some of this water exists in the oil. This water and some other chemicals serve to convert all or a portion of the metal and other materials in oil into ions. As one non-limiting example, iron (Fe) converts to Fe²⁺ and Fe³⁺ in oil 211, and aluminum (Al) converts into Al³⁺ in oil 211. A chemical or chemicals may be added to the oil to increase the conversion of metal and other materials into ions. For example, very small amounts of hydrogen peroxide (H₂O₂) have been added to oil 211 to increase the conversion of metal or other materials 214 in oil 211 into ions 216 without any noticeable effect on the performance of the oil 211 or mechanical system 200. Metal or other materials 214 in oil 211 of a mechanical system 200 may include one or more of the following: silver, aluminum, gold, barium, beryllium, bromine, cadmium, calcium, cerium, cobalt, chromium, copper, iron, lithium magnesium, manganese, molybdenum, sodium, nickel, lead, silicon, tantalum, tin, titanium, vanadium, zinc, zirconium; and the ions 216 in oil 211 of a mechanical system include one or more of the following atoms or polyatomic molecules: Ag⁺, Al³⁺, Au⁺, Au³⁺, Ba²⁺, Be²⁺, Br⁻, Ca²⁺, Ce³⁺, Co²⁺, Co³⁺, Cr²⁺, Cr³⁺, Cu⁺, Cu²⁺, Fe²⁺, Fe³⁺, Li⁺, Mg²⁺, Mn²⁺, Mn³⁺, Mo⁶⁺, Na⁺, Ni²⁺, Ni³⁺, Pb²⁺, Pb⁴⁺, SiO₄⁴⁻, Ta⁵⁺, Sb²⁺, Sb⁵⁺, Sn²⁺, Sn⁴⁺, Ti³⁺, Ti⁴⁺, V³⁺, V⁵⁺, Zn²⁺, and Zr⁴⁺. Except for nine elements (boron, carbon, silicon, helium, neon, argon, krypton, xenon and radon), all elements have at least one ionic component, any of which can be detected and measured in oil by some embodiments; and boron, carbon and silicon each have polyatomic ions that can be detected and measured in oil by some embodiments. In addition, numerous other polyatomic compounds are ionic, any of which can be detected and measured by some embodiments. Some embodiments discussed herein detect only iron, some detect only aluminum, some detect only chromium, and some detect a combination. Some embodiments can detect one ion or material or a combination of ions or other materials.

Under manufacturer-specified operating parameters of the mechanical system 200 and under hydrodynamic lubrication, no mechanical parts of the mechanical system 200 should theoretically touch or rub against each other. Therefore, theoretically, if the mechanical system 200 is operating under manufacturer-specified parameters and the oil 211 has not degraded, and if there is no contamination of metal or other materials 214 or ions 216 into the mechanical system 200, there should be no theoretical increase in the concentration of metal or other materials 214 or ions 216 in the oil 211. However, in reality, the mechanical parts of a mechanical system 200 rub against each other and/or corrode, causing wear and resultant deposition of metal and other materials in the oil 211. Further, if the mechanical system 200 is operating outside the manufacturer-specified operating parameters, the amount of rubbing and/or corrosion of mechanical parts increases, and the amount of metal or other materials 214 in the oil 216 increases. Some examples of operating outside the manufacturer-specified operating parameters include shafts and/or bearings out of alignment, operating at too high of loads or vibrations, operating a too high or too low temperature, and operating under poor lubrication viscosity. In addition, rubbing of mechanical parts occurs during startup of some mechanical systems.

In some situations, the ions or other materials 216 interact, chelate or bind 220 with the photo-responsive chemicals 218 in oil, changing the optical properties of these molecules. In some embodiments, if some of the effect of this binding is to increase or decrease the yield of photons for each drop in energy of the excited molecules, the fluorescence intensity increases or quenches respectively. If some of the effect of this binding is to change the energy of the relaxed singlet excited state (see S₁ 110 in FIG. 1), the wavelength of fluorescence for these molecules can shift. Until full chelation has occurred, some of the photo-responsive chemicals may bind to ions 220 and some of the photo-responsive chemicals may not bind to ions 218. As a result, an increase or quenching of fluorescence intensity and/or a shift in fluorescence wavelengths may be gradual, permitting a measurement of the degree of chelation and as a result a measurement of the concentration of ions and other materials in oil.

Some or all of the photo-responsive chemicals (both bound to ions 220 and unbound 218) may be excited into higher energy states by excitation sources 222. In some circumstances, the higher energy state is the same or similar for photo-responsive chemicals bound to ions 220 and unbound 218. Still, in some circumstances, the higher energy state is different for photo-responsive chemicals bound to ions 220 and unbound 218. Therefore, excitation energy hν₁ 236 may include a narrow wavelength distribution, such as from a laser source 222. Some of the advantages of a narrow wavelength distribution of light source 222 is prevention of overlap into the fluorescence emission energies hν₂ 238, resulting in lower signal-to-noise ratio.

In some situations, excitation energy hν₁ 236 comprises a slightly broader wavelength distribution, such as from a lower-cost light emitting diode source 222. If such a the slightly broader wavelength distribution causes too much noise in the emission energy spectra hν₂ 238, a filter (before excitation energy hν₁ 236 and/or after fluorescence emission energy spectra hν₂ 238) can be used. Excitation energy hν₁ 236 may comprise a combination of different wavelengths and/or distributions of light from lasers and/or light-emitting diodes. Excitation sources and “electromagnetic sources” 222 may include without limitation one or more of the following: light and optical sources (including lasers and light emitting diodes), ultraviolet sources, infrared sources, x-ray sources, gamma-ray sources and other nuclear energy sources. Excitation energies 236 may be intensity modulated over time (including sinusoidal, pulse and/or otherwise), which has been found under certain circumstances to increase the quantum yield, fluorescence intensity, and/or sensitivity of the fluorophores.

In some situations, the photo-responsive chemicals 218 change color when they interact with ions 220. Light sources 222 may be broad spectrum light sources that absorb, reflect or transmit color from the photo-responsive chemical 218 and/or 220. Light sources 222 may be powered and/or controlled by power sources 224 through power connections 226 and/or control connections 228. Alternately, power sources 224 and/or light sources 222 may be powered and/or controlled by central computer/display circuits 234 of the mechanical system 200 through power connections 230 and/or control connections 232. As one non-limiting example, the central computer/display circuit 234 may be the onboard diagnostic/control computer (OBD) of an automobile and the computer/display circuit 234 may be the “check engine” light on the dashboard of an automobile.

As mentioned above, optical properties and/or color energy spectrums hν₂ 238 may depend on several parameters, including excitation energies hν₁ 236, photo-responsive chemicals 218, ions 216 in oil 211, and the percentage of photo-responsive chemicals 218 that have chelated and/or interacted 220 with ions 216 in the oil. Given that the excitation energies hν₁ 236 and the types and properties of photo-responsive chemicals 218 can be controlled, the optical properties and/or color energy spectrums hν₂ 238 may be used to determine the types and/or concentrations of ions in oil.

Optical properties and/or color energy spectrums hν₂ 238 may be detected by the photo-detector circuit 240. In some embodiments, the photo-detector circuit 240 can measure optical properties and/or color energy spectrums hν₂ 238, including wavelength, intensity, polarity, fluorescence lifetime, spectral width, ratios of intensity, wavelengths or widths of peaks of a plurality of spectrums, color, spectral centroid, center, full width half maximum, full width quarter maximum, integral, or 90% bandwidth. The photo-detector circuit(s) 240 may include one or more of the following: (i) a photo-diode, (ii) a spectrometer, (iii) an optical filter (fixed or tunable), (iv) a photo-resistor, (v) a photo-multiplier, (vi) a prism or diffraction grating, and/or (vii) a charge-coupled device (CCD).

Photo-detector circuits 240 may be powered and/or controlled by power sources 242 through power connections 246 and/or control connections 248. Power sources 242 and/or photo-detector circuits 240 are powered, used and/or controlled by interpreting/measuring circuits 244 comprising one or more of the following: (i) a microcontroller with look-up table to convert the optical measurements into ion concentrations, metal or other material concentrations, oil condition, engine condition, or contamination, (ii) a microcontroller with formulae to convert the optical measurements into of ion concentrations, metal or other material concentrations, oil condition, engine condition, or contamination, or (iii) analog circuits to condition the optical measurements and/or to convert the optical measurements into one or more of the following: ion concentrations, metal or other material concentrations, oil condition, engine condition, or contamination.

As used in this specification and the appended claims, words such as “convert”, “converting,” “measure” or “measuring” include, without limitation, one or more of the following: (i) converting data, (ii) interpreting data, (iii) comparing data to previously stored data or reference material, (iv) comparing data to previously stored data in a look-up table, or (v) using an equation or other statistical or mathematical formulae to convert data.

A function or functions of the interpreting/measuring circuits 244 may be performed by the computer/display system 234. Power sources 242 and/or photo-detector circuits 240 and/or interpreting/measuring circuits 244 may be used, powered and/or controlled by central computer/display systems 234 of the mechanical system 200 through power connections 254 and/or control connections 256. In one non-limiting example, the central computer/display system 234 may be the onboard diagnostic/control computer (OBD) of an automobile and the computer/display system 234 may also be the “check engine” light on the dashboard of an automobile. Such alarms or signals may take one or more forms, including an analog signal; frequency, voltage or current level; digital signal or data (showing among other things concentrations of ions and or materials in oil); and/or an indication to the mechanical system's computers.

All or part of the photo-excitation and photo-detection circuits (222-256) can be located inside the mechanical system in the oil 211 and/or inside the mechanical enclosure 202. If some or all of the photo-excitation and photo-detection circuits (222-256) are located inside the mechanical system in the oil 211 and/or inside the mechanical enclosure 202, some or all of the sight glass fixture (204-210) may become unnecessary.

When photo-responsive chemicals 218 interact with ions 216, photo-responsive chemicals 218 change the time required to be excited (104 in FIG. 1), undergo the Stokes shift (108 in FIG. 1) and then relax back to its ground state (114 in FIG. 1), known as “fluorescence lifetime.” This change in fluorescence lifetime of photo-responsive chemicals can be used to detect and measure ion concentrations in the oil of mechanical systems. Interpreting/measuring circuits 244 and computer/display circuits 234 may measure the fluorescence lifetimes of photo-responsive chemicals in numerous ways, including one or more of the following:
Computer/display circuits 234, power sources 224 and/or light sources 222 emit one or more short pulses, infrequent pulses, or periodic pulses of electromagnetic excitation energy 236 and computer/display circuits 234 (through interpreting/measuring circuits 244, power sources 242 and/or photo-detector circuits 240) may measure the time difference between emitted electromagnetic excitation energy/energies 236 and returning electromagnetic fluorescent emission energy/energies 238.
Computer/display circuits 234 and/or interpreting/measuring circuits 244 consist of, among other things, phase comparator circuits, measuring the phase difference between at least two electrical signals of comparable frequencies, and output electrical signals indicative of the phase differences between these input signals. Computer/display circuits 234, power sources 224 and/or excitation sources 222 may emit electromagnetic energies 236 of time-varying intensities (including sinusoidal), which are also provided to phase comparator circuits, and computer/display circuits 234 (through interpreting/measuring circuits 244, power sources 242 and/or photo-detector circuits 240) input the time-varying electrical signals received from the fluorescent energies 238 into the phase comparator circuits, which then output the phase differences (indicative of the fluorescence lifetimes of photo-responsive chemicals) into interpreting/measuring circuits 244 and computer/display circuits 234.

As ions increasingly bind with photo-responsive chemicals 220 in the oil 211, excited with excitation energies 236, the lifetimes of the fluorescent emission energies 238 changes, which according to the description above, is converted into concentrations of ions in the oil 211 by the interpreting/measuring circuit 244 and/or computer/display circuit 234. Examples of long-lifetime fluorophores include, without limitation, certain lanthanides and more specifically platinum(II) octaethylporphyrin ketone, which has the additional benefit in dark oil of fluorescing in the near infrared.

For purposes of this specification and the appended claims, “dark oil” means oil that has been oxidized and/or contains soot (including carbon and/or sulfur) to the point that the oil is significantly absorbing in the ultraviolet and visible spectrum up to about 600 nm at which point the absorbance decreases logarithmically.

Oxidation of the oil 211 can occur in various ways, including high temperature processes within the mechanical system 200. In addition, soot can be introduced to the oil 211 in various ways, including the combustion processes (blow-by) from an internal combustion engine. Fluorophores that absorb and/or fluoresce at wavelengths greater than about 600 nm can overcome the problem of dark oil absorbing excitation energy/energies 236 and/or fluorescent emission energy/energies 238 at shorter wavelengths. Examples of fluorophores that absorb and/or emit in the electromagnetic spectrum greater than about 600 nm include cyanines and their derivatives and platinum(II) octaethylporphyrin ketone. In some circumstances, these fluorophores that absorb and/or emit in the electromagnetic spectrum greater than about 600 nm are combined with other atoms and/or molecules that chelate to the metal or other material ions 216 of interest in oil 211 such that when the chelator attaches to a metal or other material ion 216 of interest, the chemical, optical and/or electrical change in the chelator is transferred to the fluorophore which then changes its optical properties, which in turn are detected according to systems or methods disclosed herein.

When photo-responsive chemicals 218 interact with ions 216, photo-responsive chemicals 218 remain in the excited state (106 or 110 in FIG. 1) for an extended period of time before relaxing back to a ground state (114 in FIG. 1). Lanthanides, as an example, can exhibit relatively long time periods in excited states, on the order of milliseconds or greater. Integrating and receiving all of these fluorescence emissions over a period of time for these photo-responsive chemicals can be used to detect and measure ion concentrations in the oil of mechanical systems, known as “time-resolved fluorescence”. Interpreting/measuring circuit(s) 244 and computer/display circuit(s) 234 may measure the time-resolved fluorescence of photo-responsive chemicals in the following way: computer/display circuit(s) 234, power source(s) 224 and/or light source(s) 222 emit short pulses, infrequent pulses, or periodic pulses of electromagnetic excitation energy 236 and computer/display circuit(s) 234 (through interpreting/measuring circuit(s) 244, power source(s) 242 and/or photo-detector circuit(s) 240) measure and aggregate the returning electromagnetic fluorescent emission energy/energies 238 over a fixed time, polarity, and/or wavelength band; and the number of photons detected over time is used to measure the concentration of ions in the oil. As ions increasingly bind with photo-responsive chemicals 220 in the oil 211, which are then excited with excitation energy/energies 236, the number of photons collected or integrated over time from the fluorescent emission energy/energies 238 changes, which as described above, is then converted into concentrations of ions in the oil 211 by the interpreting/measuring circuit 244 and/or computer/display circuit 234.

When photo-responsive chemical(s) 218 interact with ions 216, photo-responsive chemical(s) 218 change intensity of the polarity of electromagnetic energy fluoresced 238. This change in polarity of fluorescent electromagnetic energy 238 can be used to detect and measure ion concentrations in the oil of mechanical systems. Interpreting/measuring circuit(s) 244 and computer/display circuit(s) 234 a change in polarity/polarities of the fluorescent electromagnetic energy in the following way: polarization filter(s), which allow electromagnetic energy of polarities to pass to photo-detector circuit(s) 240, may be placed between fluorescent electromagnetic energy/energies 238 and the photo-detector circuit(s) 240. As ions increasingly bind with photo-responsive chemicals 220 in the oil 211, which are then excited with excitation energy/energies 236, the polarity of the fluorescent emission energy/energies 238 changes, which then changes the intensities of fluorescent emission energy/energies 238 transmitted through the polarization filter(s), where changed intensity is then detected by the photo-detector circuit 240 and power source 242 and converted into concentrations of ions in the oil 211 by the interpreting/measuring circuit 244 and/or computer/display circuit 234.

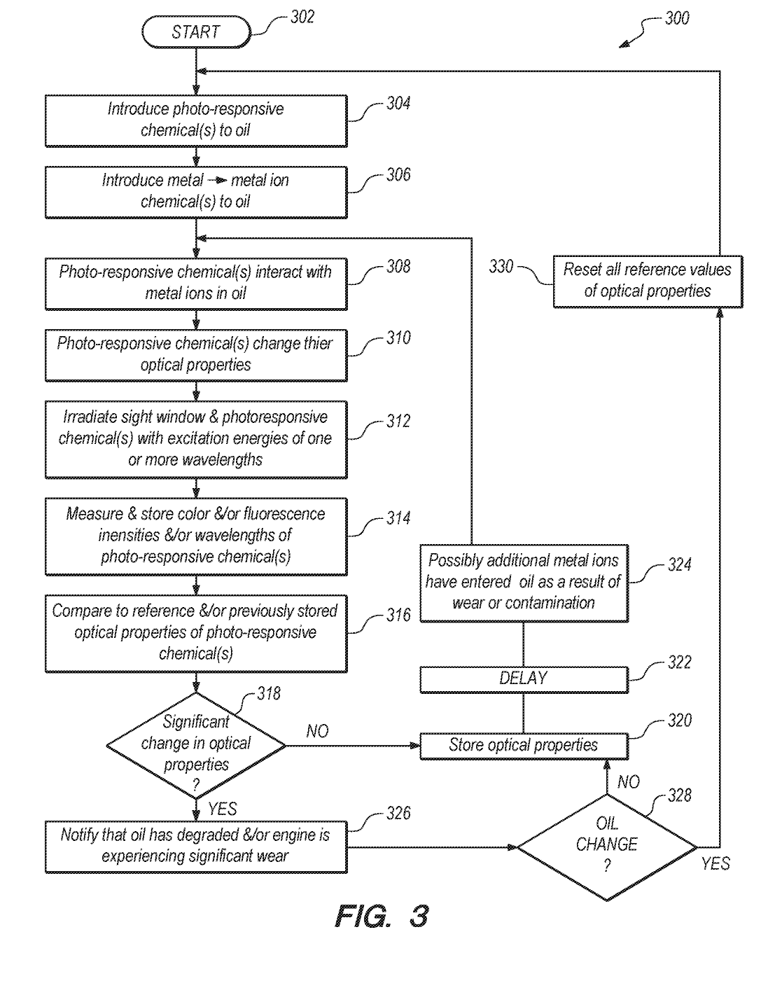

Methods 300 of measuring the concentration of materials in oil are shown in FIG. 3 and start at point 302, introducing photo-responsive chemical(s) to oil in the mechanical system 304. As noted above, some aspects of the present design introduce the photo-responsive chemicals directly into the oil. In some circumstances, 8-hydroxyquinoline, as just one of many photo-responsive chemicals, mixes readily in oil. 8-hydroxyquinoline normally ships as a solid. At approximately 125 degrees Fahrenheit, 8-hydroxyquinoline mixes and/or dissolves in oil. Further, 8-hydroxyquinoline at around 200 ppm may remain mixed and/or dissolved in oil even after cycling and/or holding the oil at temperatures up to 350 degrees Fahrenheit and down to 20 degrees Fahrenheit. At up to 200 ppm, 8-hydroxyquinoline mixed and/or dissolved in fresh oil may not be detectable in the oil to the naked eye, but such oil and/or 8-hydroxyquinoline fluoresces brightly under about 365 nanometer (nm) light. 8-hydroxyquinoline is a robust photo-responsive chemical that mixes readily in oil and remains mixed and/or dissolved under normal operating parameters of a mechanical system, including an internal combustion engine.

As discussed above, over time (under poor lubrication conditions, or problems with the mechanical system, or continual contamination or manufacturer-specified operating parameters), mechanical systems introduce metals and/or other materials to the oil in the mechanical system 306, which value and/or rate of introduction of such metals and/or other materials in the oil is indicative of oil condition, contamination, mechanical system wear and/or problems with the mechanical system, thus predicting with some accuracy the potential failure of the mechanical system.

Metals or other materials in the oil ionize 306. Additional chemicals may be introduced to the oil to increase the rate and/or level of conversion of metals or other materials in the oil to metal ions and other material ions. Further, in some situations, the concentrations of metals or other materials in oil can be measured, computed or interpreted from the concentrations of metals or other material ions measured in oil. As one non-limiting example, for any given mechanical system, oil can be sampled and measured using traditional laboratory methods to assess the concentrations of oil and other materials in the sample. In some situations, concentrations of oil and other material ions in the same sample can be measured to determine the mathematical and/or statistical relationship between the concentrations of metal and other materials to the concentrations of metals and other material ions. These mathematical and/or statistical relationships between metals and materials and their ions in oil can be stored and/or used to calibrate some of the embodiments disclosed herein for future measurement of the concentrations of metals or other materials in the oil of mechanical systems.

As a result of the introduction of metal ions and other material ions in the oil, photo-responsive chemicals in the oil may interact with the metals and other material ions 308. As one non-limiting example, in some situations, 8-hydroxyquinoline may be a strong chelator to iron ions, aluminum ions, and some other metal or material ions, meaning that 8-hydroxyquinoline binds strongly to these metal or material ions. Further as discussed above, the optical properties of these photo-responsive chemicals may change 310 as a result of these interactions. As one non-limiting example, 8-hydroxyquinoline fluoresces brightly at about 510 nm in fresh oil, and the intensity of fluorescence and the fluorescence peak wavelength decrease for increased concentrations of iron in oil.

These chemicals are photo-responsive because their optical properties change in response to irradiated electromagnetic energy as a result of interactions of these photo-responsive chemicals with metal ions or other material ions. Therefore, electromagnetic energy may be irradiated on these photo-responsive chemicals, both (i) photo-responsive chemicals that have interacted with metal ions or other material ions in the oil and (ii) photo-responsive chemicals that have not interacted with metal or other material ions in the oil 312. As discussed above, the photo-responsive chemical molecules that have interacted with metal ions or other material ions in the oil change their optical properties, including one or more of the following changes in optical properties:
Color in response to irradiated light of a known spectrum or temperature;
Fluorescence intensities at wavelengths in response to excitation energies at certain wavelengths; and/or
Ratios of fluorescence intensities of a plurality of wavelengths in response to excitation at certain wavelengths.

Because these changes occur in only the photo-responsive chemicals that have interacted with metal ions or other material ions in the oil and not the other photo-responsive chemicals in the oil, these changes are gradual and change as more and more metals or other materials are introduced to the oil. This means that detecting and/or measuring these gradual changes in optical properties can therefore measure the concentrations and rate of change of concentrations (i.e. trend data) of metals or other materials in oil.

The light, which is fluoresced from, reflected by, absorbed by, or transmitted through the photo-responsive chemicals in the oil (both photo-responsive chemicals that have interacted with metal ions or other material ions in the oil and photo-responsive chemicals that have not interacted with metal ions or other material ions in the oil), may be captured, measured and stored 314. Further, as discussed above, in some embodiments, these optical properties are stored as a function of time as rate and trend data 314. In addition, as discussed above, in some embodiments, these optical properties and rate and trend data are converted to concentrations of metals or other materials in the oil, either directly and/or indirectly as concentrations of metal ions or other material ions in the oil and such concentrations and rate and trend data are stored for later use 314.

At this point, some embodiments of the present design have measured and stored value, rate, and/or trend data for concentrations of metals or other materials in oil in a mechanical system. In some embodiments, these data are then compared to previously stored data, reference material, and/or stored mathematical or statistical formulae 316, look-up translation data or other information to determine if there is an alarm or signal to generate 318, including one or more of the following signals and/or alarms:
Oil condition has deteriorated;
Oil condition deterioration has accelerated;
Oil change is needed;
Wear in the mechanical system;
Wear in the mechanical system has accelerated;
Contamination has occurred;
Contamination has accelerated; or
Probability of mechanical failure has increased.

Alarms or signals may take different forms, including an analog signal or level (including light, frequency, voltage and/or current), digital signal or data, or an indication to a mechanical system's computer.

If no alarm and/or signal occurs, the current data are stored with other previously stored data 320. If an alarm or signal occurs, the owner or operator of the mechanical system may receive the alarm or notification 326 and decide whether an oil change is necessary 328. If an oil change is necessary, all or a portion of the previously measure and stored data are reset 330, and the process is repeated, starting at point 304. However, if an oil change is not necessary, the oil remains in the mechanical system along with the metal or other materials, metal ions, or other material ions, and the photo-responsive chemicals, such that the previously stored data 320 are still relevant and stored.

The methods described above may not occur continuously or at a high frequency. They can occur once or more about every second, minute, hour, or even day depending on the operating parameters of the mechanical system 322. During this delay period 322, additional metal or other materials or metal ions or other material ions may have been introduced to the oil in the mechanical system 324 as the result of operating the mechanical system. In some embodiments, the process of reading the changes in the optical properties and correlating these values and trends to machine or oil condition (308-330) may then be repeated.

Some of the steps shown in FIG. 3 can be changed or deleted. The present design may be implemented as a portable or handheld system (known herein as a “measurement system”) that is separate from the mechanical system. As a non-limiting example, for a portable or handheld system, the oil (possibly with ions) may be sampled from the mechanical system, the ion concentration measured or assessed remotely from the mechanical system, and in this situation, step 306 occurs before step 304. One or more of the photo-responsive chemicals may be attached to a rigid mechanical structure (either directly or indirectly through a solid chemical such as silica gel) such as a dipstick or the inside of a glass sampling vial. The photo-responsive chemicals may be exposed to the ions in the oil by dipping the dipstick into the oil, either directly in the mechanical system or in an oil sample, or the oil may be placed inside the sampling vial.

The graph of FIG. 4 shows the relative light intensity versus wavelength (spectrograph) of various concentrations of iron in oil using certain of the aspects and devices described herein. 8-hydroxyquinoline was added to oil at 200 ppm. Then, an iron, oil-based standard solution at 1,000 ppm from Alfa Aesar (P/N 14220) was added to the 8-hydroxyquinoline-iron solution at various amounts to achieve 100 milliliter samples at iron-oil concentrations from 3 ppm to 160 ppm. An excitation energy of 365 nm wavelength light 402 was irradiated on the 100 milliliter samples of this 8-hydroxyquinoline-oil solution at concentrations ranging from 3 ppm to 160 ppm. Fresh motor oil has concentrations of iron at about 3 ppm, which fluorescence peaks at about 510 nm 404 with 8-hydroxyquinoline in the oil. Motor oil for a typical internal combustion engine has iron concentrations that range from about 3 ppm (fresh oil) to about 100 ppm (bad oil, contamination and/or engine trouble). Shown on the graph of FIG. 4 are light intensities for concentrations of iron in oil for 3 ppm 404, 10 ppm 406, 20 ppm 408, 40 ppm 410, 80 ppm 412 and 160 ppm 414 using some of the embodiments disclosed herein. This shows that in some embodiments, 8-hydroxyquinoline in oil fluoresces brightly for 3 ppm iron-oil concentrations at about 510 nm peak wavelength. Further, in some embodiments, the fluorescence intensity quenches logarithmically as the iron concentration in the oil increases; and the peak fluorescence wavelength shifts from about 510 nm to 497 nm as the iron concentration in oil increases. This shows that iron concentrations can be effectively detected and measured using a dilute 8-hydroxyquinoline-in-oil solution.

Further, a dilute 8-hydroxyquinoline-in-oil solution increases in fluorescence intensity at about 510 nm peak wavelength in increasing concentrations of aluminum ions (Al³⁺) in oil. In some embodiments, a dilute 8-hydroxyquinoline-in-oil solution can not only detect concentrations of iron and aluminum metal ions in oil (by detecting and monitoring the level of fluorescence decrease or increase respectively) but may also detect which of these two metals is in the oil (by detecting and monitoring the fluorescence decrease or increase). In some embodiments, a logarithmic, mathematical equation may be produced which considers the decrease in fluorescence intensity of oil and 8-hydroxyquinoline and outputs a concentration of iron in the oil.

Manufactured motor oil fluoresces at about 415 nm (major peak) and about 490 nm (minor peak), although this oil fluorescence intensity is about 75% less than with 200 ppm 8-hydroxyquinoline added the same oil. This is known as auto fluorescence. Further, these oil fluorescence peaks decrease with increased concentrations of iron ions in the oil. Oil auto fluorescence peaks can be measured to determine the metal or other material ion concentrations in oil. Oil auto fluorescence peaks can also be measured to determine the concentrations of contaminants or other materials in oil. Oil auto fluorescence peaks may be measured in conjunction with the fluorescence peaks of photo-responsive chemicals to increase the accuracy, range or resolution of measuring concentrations of ions in oil.

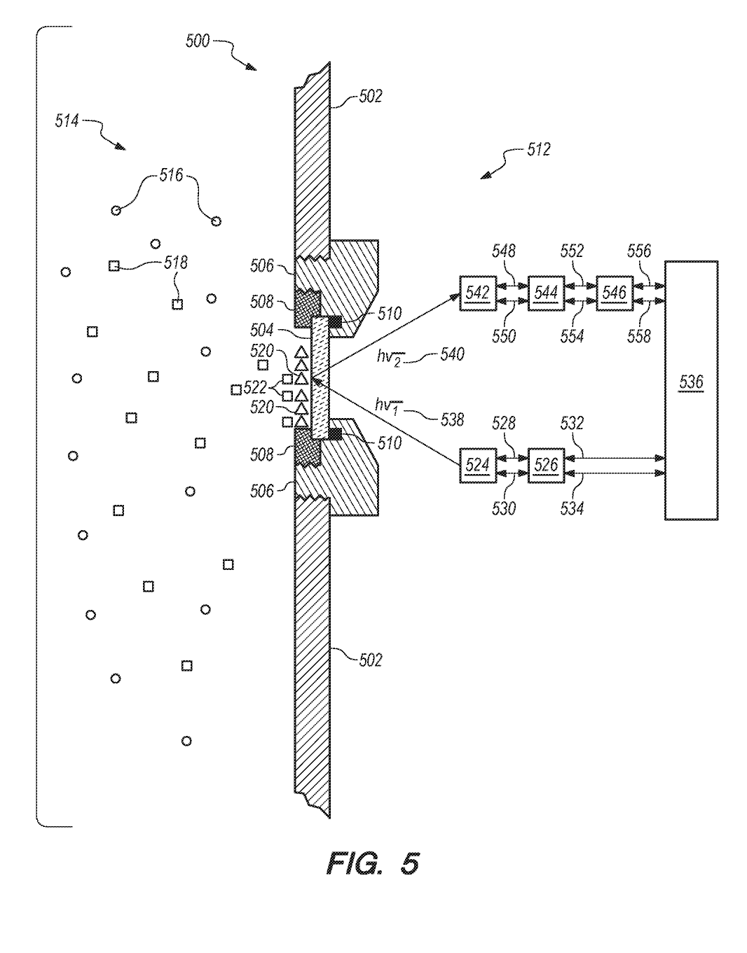

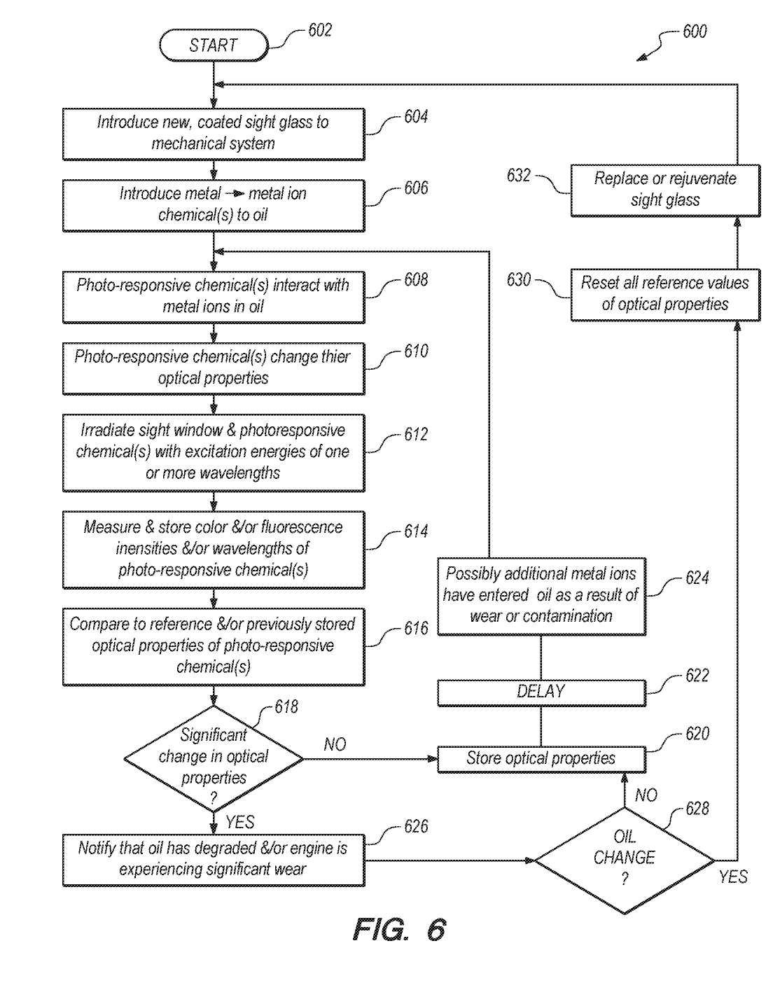

FIG. 5 shows a mechanical arrangement that may be used to measure the concentration of ions and other materials in oil in a mechanical system 500, which includes a cutaway side view of the mechanical enclosure 502 and sight glass fixture (504-510). Embodiments represented by FIG. 5 are similar to some embodiments represented by FIG. 2 with one or more of the following exceptions:
Whereas photo-responsive chemicals 218 in FIG. 2 are introduced to, dissolved in, and/or suspended in the oil 211, the photo-responsive chemicals 520 in FIG. 5 are attached to a mechanical structure 504 and then exposed to the oil and the ions and other materials in the oil.
Whereas excitation sources 222 in FIG. 2 excite photo-responsive chemicals 218 in the oil 211, excitation sources 524 in FIG. 5 excite photo-responsive chemicals 520 attached to the mechanical structure 504 exposed to the oil, as well as ions and other materials in the oil.
Whereas photo-responsive chemicals 218 in FIG. 2 are refreshed by replacing the photo-responsive chemicals in the oil 211, usually after an oil change (see also point 304 in FIG. 3), photo-responsive chemicals 520 in FIG. 5 may be refreshed by (i) replacing the photo-responsive chemicals 520 and the mechanical structure 504 in which they are attached and/or (ii) rejuvenating the photo-responsive chemicals 520 by introducing the photo-responsive chemicals 520 and/or the mechanical structure 504 with a strong chelator, such as the chemical CN⁻ as one non-limiting example (see also point 632 in FIG. 6).

Some designs include a combination of some of the embodiments in FIG. 2 and some embodiments in FIG. 5. As one non-limiting example, photo-responsive chemicals are introduced to the oil 211 as in FIG. 2, whereas some photo-responsive chemicals are attached to a mechanical structure 504 in and/or on the mechanical enclosure 502 as in FIG. 5. In this example, the photo-responsive chemicals in the oil detect the concentrations of different ions or other materials from the photo-responsive chemicals attached to the mechanical structure 504.

Most oil-lubricated mechanical systems 500 are encased in a mechanical enclosure 502 that holds oil 514. In some systems, the oil 514 is recirculated though the mechanical system 500 via an oil pump and circulation channels, or the oil 514 is agitated and mixed by the motion of the parts of the mechanical system 500 itself.

Some embodiments include a sight glass fixture (504-510). In some instances, the transparent sight glass 504 permits excitation energies hν₁ 538 from excitation sources 524 to enter the mechanical system 500 and interact with the oil 514 and/or photo-responsive chemicals 520 and/or permits electromagnetic energy hν₂ 540 to exit the oil 514 and/or photo-responsive chemicals 520 and to be detected by one or more photo-detector circuits 542. The sight glass fixture (504-510) can be custom built or obtained from commercial sources. Sight glass fixtures can take many shapes and forms, including circular or cylindrical. The sight glass fixture (504-510) can be circular and attached to the mechanical enclosure 502, which can include a block, oil pan, oil conduit, casing or oil filter cover for the mechanical system. The sight glass fixture (504-510) can be cylindrical and attached to the oil recirculating system of the mechanical system 500.

The circular sight glass fixture (504-510) typically comprises four basic parts: (i) the sight glass 504, made of various materials including fused glass or plastic depending on the operating parameters, including temperature and pressure, of the oil 514 in the mechanical system 500; (ii) sight glass fixture casing 506 upon which the sight glass 504 rests; (iii) circular gasket 510, which prevents oil 514, metal or other materials 516, ions 518 and photo-responsive chemicals 520 from escaping the mechanical system 500 and entering the exterior environment 512, including air; and (iv) a circular flange 508 which screws into the sight glass fixture casing 506 and presses against the sight glass 504 and circular gasket 510 to prevent movement of the sight glass 504 and prevents oil 514, metal or other materials 516, ions 518 and photo-responsive chemicals 520 from escaping the mechanical system 500 and entering the exterior environment 512, including the air.

Photo-responsive chemicals 520 may attach or bond to the sight glass 504, such that the sight glass 504 serves as the mechanical structure holding the one or more photo-responsive chemicals in the presence of the oil 514. The sight glass fixture (504-510) may be permanently attached to the mechanical enclosure 502. The sight glass fixture (504-510) or the sight glass 504 may be periodically replaced, including with each oil change or when at least one photo-responsive chemical attached to the sight glass 504 is fully chelated. Photo-responsive chemicals are bonded or attached to more solid chemicals, which are then attached, glued or bonded to the mechanical structure. One non-limiting example of a solid chemical in which photo-responsive chemicals can be attached is silica gel (SiO₂) such as silica gel, wide pore, 150 angstroms (350-400 meters/gram) from Alfa Aesar (P/N 42728) (CAS 63231-67-4). Certain chemicals, including fluorophores, can bond to the surface of solid chemicals such as silica gel. In this way, photo-responsive chemicals can attach to a more stable, solid chemical with a very large surface area (thereby permitting large exposure of the photo-responsive chemicals to the ions in oil) and also permit strong adhesion of the photo-responsive chemical(s) and solid chemical to the mechanical structure.

As used in this specification and the appended claims, the word “solid chemical” includes any chemical that remains in solid form under the operating or testing conditions of the mechanical system or measurement system.

Purposes for the solid chemical include (i) binding one or more photo-responsive chemicals to a determined part of the mechanical structure or measurement system or (ii) stabilizing one or more of the photo-responsive chemicals with respect to various parameters, including temperature, pressure, hydrodynamic forces, acidity, or the effects of other chemicals. The solid chemical may allow substantially only those ions of interest access to the photo-responsive chemicals, increasing the selectivity of the photo-responsive chemicals to the ions of interest. Photo-responsive chemicals may be attached to certain solid chemicals, including silica (which has very small pores and a very high ratio of surface area to volume) so as to increase the selectivity of the photo-responsive chemical to certain ions in the oil. Photo responsive chemicals may bond directly to the mechanical structure. Other photo-responsive chemicals may be used to measure the concentrations of ions in oil, including the following photo-responsive chemicals: calcein and/or calcein derivatives, cyanine and/or cyanine derivatives, rhodamine and/or rhodamine derivatives, and/or 8-hydroxyquinoline and/or 8-hydroxyquinoline derivatives.

Atoms and/or poly-atomic molecules may be added to or bound to the photo-responsive chemicals to enhance their photo-responsive properties, including selectivity to certain ions, sensitivity, fluorescence quantum yield, fluorescence intensity multiple, quenching fraction, wavelength of peaks of the fluorescence spectra, and the shifting of wavelengths of peaks of the fluorescence spectra. These enhanced photo-responsive chemicals are known as derivatives of the above described photo-responsive chemicals. As a non-limiting example, molecules may chelate to ions in oil, changing the chemical or electrical properties of the molecule(s); and this change is transferred (via resonance energy transfer or otherwise) to other molecules whereby fluorescence properties change. In the aggregate, this junction of these different types of molecules (one or more chelators and one or more fluorophores) serves as an effective sensor in some embodiments. The photo-responsive chemical may change its color in response to interactions with ions in oil. Independently or on conjunction with fluorescence, color-changing, photo-responsive chemicals serve to determine the concentration of ions in oil by measuring the gradual change in color of the photo-responsive chemicals in response to gradual changes in the concentration of ions in the oil.

In general, very small amounts of metal or other materials 516 are present in fresh oil 514 after each oil change. Fresh oil has been found to contain iron and/or aluminum up to about 3 ppm each. Although oil 514 is hydrophobic, some water and other chemicals exist in oil. In fact, up to one gallon of water can be produced for every gallon of fuel consumed in some gasoline and diesel internal combustion engines. Some of this water exists in the oil. This water and some other chemicals serve to convert all or a portion of the metal or other materials in oil into ions. As one non-limiting example, iron (Fe) converts to Fe²⁺ and Fe³⁺ in oil 514, and aluminum (Al) converts into Al³⁺ in oil 514. Chemicals may be added to the oil to increase the conversion of metal and other materials and/or molecules into ions. For example, very small amounts of hydrogen peroxide (H₂O₂) have been added to oil 514 to increase the conversion of metal and other materials 516 in oil 514 into ions 518 without any noticeable effect on the oil 514 or mechanical system 500.

Metals and other materials 516 found in oil 514 of a mechanical system 500 may include one or more of the following: silver, aluminum, gold, barium, beryllium, bromine, cadmium, calcium, cerium, cobalt, chromium, copper, iron, lithium magnesium, manganese, molybdenum, sodium, nickel, lead, silicon, tantalum, tin, titanium, vanadium, zinc, zirconium; and the ions 518 in oil 514 of a mechanical system include one or more of the following atoms or polyatomic molecules: Ag⁺, Al³⁺, Au⁺, Au³⁺, Ba²⁺, Be²⁺, Br⁻, Ca²⁺, Ce³⁺, Co²⁺, Co³⁺, Cr²⁺, Cr³⁺, Cu⁺, Cu²⁺, Fe²⁺, Fe³⁺, Li⁺, Mg²⁺, Mn²⁺, Mn³⁺, Mo⁶⁺, Na⁺, Ni²⁺, Ni³⁺, Pb²⁺, Pb⁴⁺, SiO₄⁴⁻, Ta⁵⁺, Sb²⁺, Sb⁵⁺, Sn²⁺, Sn⁴⁺, Ti³⁺, Ti⁴⁺, V³⁺, V⁵⁺, Zn²⁺, and Zr⁴⁺. Except for nine elements (boron, carbon, silicon, helium, neon, argon, krypton, xenon and radon), all elements have at least one ionic component, any one or more of which can be detected and measured in oil by some embodiments; and boron, carbon and silicon each have polyatomic ions that can be detected and measured in oil by some of the embodiments disclosed herein. In addition, numerous polyatomic compounds are ionic, any one or more of which can be detected and measured by some of the embodiments disclosed. However, some embodiments of the current design detect only iron, while some embodiments detect only aluminum, while further some embodiment detect only chromium, while still other embodiments detect a combination. Some embodiments can detect one or a combination of ions.

Under manufacturer-specified operating parameters of the mechanical system 500 and under hydrodynamic lubrication, no mechanical parts of the mechanical system 500 should theoretically touch or rub against each other. Therefore, theoretically, if the mechanical system 500 is operating under manufacturer-specified parameters and the oil 514 has not degraded, and there is no contamination of metal or other materials 516 or ions 518 into the mechanical system 500, then there should be no theoretical increase in the concentration of metal or other materials 516 or ions 518 in the oil 514. However, in reality, the mechanical parts of a mechanical system 500 rub against each other, even under normal operating parameters, causing wear and subsequently metal and other materials in the oil 514. Further, if the mechanical system 500 is operating outside the manufacturer-specified operating parameters, the amount of rubbing of mechanical parts increases and the amount of metal or other materials 516 in the oil 514 increases. Some examples of operating outside the manufacturer-specified operating parameters include shafts and/or bearings out of alignment, operating at too high of loads or vibrations, operating a too high or too low temperature, and operating under poor lubrication viscosity.

These ions 518 interact, chelate or bind 522 with one or more of the photo-responsive chemicals 520 in the presence of oil 514, changing the optical properties of these molecules. If some of the effect of this binding is to increase or decrease the yield of photons for each drop in energy of the excited molecules, the fluorescence intensity increases or quenches respectively. If some of the effect of this binding is to change the energy of the relaxed singlet excited state (see S₁ 110 in FIG. 1), the wavelength of fluorescence for these molecules typically will shift. Until full chelation has occurred, some of the photo-responsive chemicals may be bound to ions 522 and some of the photo-responsive chemicals will not be bound to ions 520. As a result, an increase or quenching of fluorescence intensity and/or a shift in fluorescence wavelengths may be gradual, thereby permitting a measurement of the degree of chelation and therefore a measurement of the concentration of ions in oil.

Some or all of the photo-responsive chemicals (both bound to ions 522 and unbound 520) are excited into higher energy states by excitation sources 524. The higher energy state is the same for photo-responsive chemicals bound to ions 522 and unbound 520. Still, the higher energy state is different for photo-responsive chemicals bound to ions 522 and unbound 520. Therefore, excitation energy hν₁ 538 may comprise a narrow wavelength distribution, such as from a laser source 524. Some of the advantages of a narrow wavelength distribution of light source 524 is prevention of overlap into the fluorescence emission energy hν₂ 540, which results in a lower signal-to-noise ratio. Excitation energy hν₁ 538 comprises a slightly broader wavelength distribution, such as from a lower-cost light emitting diode source 524. If the slightly broader wavelength distribution, such as from a lower-cost light emitting diode source 524, causes too much noise in the emission energy spectra hν₂ 540, a filter (before excitation energy hν₁ 538 and/or after fluorescence emission energy hν₂ 540) can be used. Excitation energy hν₁ 538 may comprise a combination of different wavelengths and/or distributions of light from lasers and/or light-emitting diodes. Excitation energies 538 may be intensity modulated over time (including sinusoidal, pulse and/or otherwise), which may under certain circumstances increase the quantum yield, fluorescence intensity and/or sensitivity of the photo-responsive chemicals.

The photo-responsive chemicals 520 may change color when they interact with ions 522. Light sources 524 may be broad spectrum light sources that absorb, reflect or transmit color from the photo-responsive chemicals 520 and/or 522.

Light sources 524 are powered and/or controlled by power sources 526 through power connections 528 and/or control connections 530. Power sources 526 and/or light sources 524 may be powered and/or controlled by central computer/display circuits 536 of the mechanical system 500 through power connections 532 and/or control connections 534. As one non-limiting example, the central computer/display circuit 536 is the onboard diagnostic/control computer (OBD) of an automobile and the computer/display circuit 536 is also the “check engine” light on the dashboard of an automobile.

Optical properties and/or color energy spectrums hν₂ 540 are dependent on several parameters, including excitation energies hν₁ 538, photo-responsive chemicals 520, ions 518 in oil 514, and the percentage of photo-responsive chemicals 520 that have chelated and/or interacted 522 with ions 518 in the oil. In some embodiments, given that the excitation energies hν₁ 538 and the types and properties of photo-responsive chemicals 520 can be controlled, the optical properties and/or color energy spectrums hν₂ 540 are used to determine the types and concentrations of ions in oil.

Optical properties and/or color energy spectrums hν₂ 540 are detected by the photo-detector circuit 542. The photo-detector circuit can measure optical properties and/or color energy spectrums hν₂ 540, including wavelength, intensity, polarity, fluorescence lifetime, spectral width, ratios of intensity, wavelengths or widths of peaks of a plurality of spectrums, color, spectral centroid, center, full width half maximum, full width quarter maximum, integral, or 90% bandwidth. The photo-detector circuits 536 may include one or more of the following: (i) photo-diode, (ii) spectrometer, (iii) optical filter (fixed or tunable), (iv) photo-resistor, (v) photo-multiplier, (vi) prism or diffraction grating, and/or (vii) charge-coupled device (CCD).

Photo-detector circuits 542 may be powered and/or controlled by power sources 544 through power connections 548 and/or control connections 550. Power sources 544 and/or photo-detector circuits 542 are powered, used and/or controlled by interpreting/measuring circuits 546. Interpreting/measuring circuits 546 may include one or more of the following: (i) microcontroller with look-up table to convert the optical measurements into ion concentrations, metal or material concentrations, oil condition, engine condition, and/or contamination, (ii) microcontroller with formulae to convert the optical measurements into ion concentrations, metal or material concentrations, oil condition, engine condition, and/or contamination, and (iii) analog circuits to condition the optical measurements and/or convert the optical measurements into ion concentrations, metal or other material concentrations, oil condition, engine condition, and/or contamination. Functions of the interpreting/measuring circuits 546 may be performed by the computer/display circuit 536. Power sources 544 and/or photo-detector circuits 542 and/or interpreting/measuring circuits 546 are used, powered and/or controlled by central computer/display circuits 536 of the mechanical system 500 through power connections 556 and/or control connections 558. As one non-limiting example, the central computer/display circuit 536 is the onboard diagnostic/control computer (OBD) of an automobile and the computer/display circuit 536 is also the “check engine” light on the dashboard of an automobile.

When photo-responsive chemicals 520 interact with ions 518, photo-responsive chemicals 520 change the time required to be excited (104 in FIG. 1), undergo the Stokes shift (108 in FIG. 1) and relax back to a ground state (114 in FIG. 1), known as “fluorescence lifetime.” This change in fluorescence lifetime of photo-responsive chemicals can be used to detect and measure ion concentrations in the oil of mechanical systems. Interpreting/measuring circuits 546 and computer/display circuits 536 measure the fluorescence lifetimes of photo-responsive chemicals in numerous ways, including one or more of the following:
Computer/display circuits 536, power sources 526 and/or light sources 524 may emit one or more short pulses, infrequent pulses, or periodic pulses of electromagnetic excitation energy 538 and then computer/display circuits 536 (through interpreting/measuring circuits 546, power sources 544 and/or photo-detector circuits 542) measure the time difference between the emitted electromagnetic excitation energies 538 and the returning electromagnetic fluorescent emission energies 540.
Computer/display circuits 536 and/or interpreting/measuring circuits 546 include, among other things, phase comparator circuits that measure the phase difference between at least two electrical signals of comparable frequencies and output electrical signals indicative of the phase differences between these input signals. Computer/display circuits 536, power sources 526, and/or excitation sources 524 emit electromagnetic energies 538 of time-varying intensities (including sinusoidal), which are also inputted into phase comparator circuits and then computer/display circuits 536 (through interpreting/measuring circuits 546, power sources 544 and/or photo-detector circuits 542) input the time-varying electrical signals received from the fluorescent energies 540 into phase comparator circuits, which then output the phase difference signals (indicative of the fluorescence lifetimes of photo-responsive chemicals) into interpreting/measuring circuits 546 and computer/display circuits 536.

As ions 518 increasingly bind with photo-responsive chemicals 520 in the oil 514, which are then excited with excitation energies 538, the lifetimes of the fluorescent emission energies 540 changes, which according to the description above, is then converted into concentrations of ions 518 in the oil 514 by the interpreting/measuring circuit 546 and/or computer/display circuit 536. Examples of long-lifetime fluorophores include, without limitation, certain lanthanides and more specifically platinum(II) octaethylporphyrin ketone, which has the additional benefit in dark oil of fluorescing in the near infrared.

When photo-responsive chemicals 520 interact with ions 518, photo-responsive chemicals 520 remain in the excited state (106 or 110 in FIG. 1) for an extended period of time before relaxing back to a ground state (114 in FIG. 1). Interpreting/measuring circuits 546 and computer/display circuits 536 measure the time-resolved fluorescence of photo-responsive chemicals in the following way: computer/display circuits 536, power sources 526, and/or light sources 524 emit short pulses, infrequent pulses, or periodic pulses of electromagnetic excitation energy 538 and then computer/display circuits 536 (through interpreting/measuring circuits 546, power sources 544 and/or photo-detector circuits 542) measure and aggregate the returning electromagnetic fluorescent emission energies 540 over a fixed period of time; and the number of photons detected over a period of time may be converted into the concentration of ions in the oil. As ions increasingly bind with photo-responsive chemicals 520 in the oil 514, which are then excited with excitation energies 538, the number of photons collected or integrated over time from the fluorescent emission energy/enrgies 540 changes, which according to the time-resolved description above, is then converted into concentrations of ions 518 in the oil 514 by the interpreting/measuring circuit 546 and/or computer/display circuit 536.

When photo-responsive chemicals 520 interact with ions 518, photo-responsive chemicals 520 change the intensities of the polarity of electromagnetic energy fluoresced 540. This change in polarity of fluorescent electromagnetic energy 540 is used to detect and measure ion concentrations in the oil of mechanical systems. Interpreting/measuring circuits 546 and computer/display circuits 536 measure the changes in polarities of the fluorescent electromagnetic energy in the following way: polarization filters, which allow electromagnetic energy of certain polarities to pass to photo-detector circuits 542, are placed between the fluorescent electromagnetic energies 540 and the photo-detector circuits 542. As ions 518 increasingly bind with photo-responsive chemicals 520 in the oil 514, and are then excited with excitation energies 538, the polarity of the fluorescent emission energies 540 changes, which then changes the intensities of the fluorescent emission energies 540 transmitted through the polarization filters. The changed intensity may then be detected by the photo-detector circuit 542 and power source 544 and then converted into concentration(s) of ions 518 in the oil 514 by the interpreting/measuring circuit 546 and/or computer/display circuit 536.

Methods 600 of measuring the concentrations of materials in oil are shown in FIG. 6 and start 602 with installing a sight glass fixture to the mechanical system, which is coated with one or more photo-responsive chemicals and exposed to oil in the mechanical structure 604. One or more of the photo-responsive chemicals may bond to a solid chemical (such as silica gel, which has a very high ratio of surface area to mass), which is then attached to a rigid structure in the presence of oil. In some embodiments, 8-hydroxyquinoline attached to a mechanical structure and exposed to oil in the mechanical system, is a robust photo-responsive chemical that does not change the operating parameters of the mechanical system, including an internal combustion engine. The photo-responsive chemicals may be attached to a rigid mechanical structure (either directly or indirectly through a solid chemical such as silica gel) such as a dipstick or sampling vial. The photo-responsive chemicals may be exposed to the ions in the oil by dipping the dipstick into the oil, either directly in the mechanical system or in an oil sample or adding the oil to the sampling vial. As a non-limiting example, for a portable or handheld system, the oil (possibly with ions) may be sampled from the mechanical system, and some embodiments measure the ion concentration remotely from the mechanical system, and therefore, step 606 occurs before 604, and step 632 is deleted.

Over operating time (under poor lubrication conditions, under problems with the mechanical system, or even under manufacturer-specified operating parameters), mechanical systems introduce metals and/or other materials to the oil in the mechanical system 606, wherein value and/or rate of introduction of such materials in the oil is indicative of oil condition, oil contamination, mechanical system wear and/or problems with the mechanical system. Such readings or measurements can be used as a predictor of failure of the mechanical system.

Metals or other materials in the oil ionize 606. Additional chemicals may be introduced to the oil to increase the rate and/or level of conversion of metals or other materials in the oil to metal ions and other material ions. The concentrations of metals or other materials in oil can be measured, computed or interpreted from measuring the concentrations of metals or other material ions in oil. As one non-limiting example, for any given mechanical system, oil can be sampled and measured using traditional laboratory methods to ascertain the concentrations of oil and other materials in the sample. The concentrations of oil and other material ions in the same sample may be measured to determine the mathematical and/or statistical relationship between the concentrations of metal and other materials to the concentrations of metals and other material ions. In some circumstances, these mathematical and/or statistical relationships between metals and materials and their ions in oil can be stored and/or used to calibrate the methods and devices disclosed herein for future measurement of the concentrations of metals or other materials in the oil of mechanical systems.

As a result of the introduction of metal and other material ions in the oil, the photo-responsive chemicals in the oil interact with the metals and other material ions 608. 8-hydroxyquinoline, for example, is a strong chelator to iron ions, aluminum ions and some other metal or material ions, indicating 8-hydroxyquinoline binds strongly to these metal or material ions. Optical properties of these photo-responsive chemicals change 610 as a result of these interactions. 8-hydroxyquinoline fluoresces brightly at about 510 nm in fresh oil and the intensity of fluorescence and the fluorescence peak wavelength tend to decrease for increased concentrations of iron in oil.

These chemicals are photo-responsive because one or more of their optical properties change in response to irradiated electromagnetic energy as a result of interactions of these photo-responsive chemicals with metal ions or other material ions. Therefore, in certain aspects of the current design, electromagnetic energy is irradiated on these photo-responsive chemicals, both (i) photo-responsive chemicals that have interacted with metal ions or other material ions in the oil and (ii) photo-responsive chemicals that have not interacted with metal ions or other material ions in the oil 612. The photo-responsive chemical molecules that have interacted with metal ions or other material ions in the oil change their optical properties, including one or more of the following:
Color changes in response to irradiated light of a known spectrum or temperature;
Fluorescence intensities changes at one or more wavelengths in response to excitation at one or more wavelengths; and/or
Ratios of fluorescence intensity changes of multiple wavelengths in response to excitation at one or more wavelengths.

Because these changes occur in only the photo-responsive chemicals that have interacted with metal ions or other material ions in the oil and not the other photo-responsive chemicals in the oil, these changes are gradual and vary as more and more metals or other materials are introduced to the oil. This means that detecting and/or measuring these gradual changes in optical properties can quantify the concentrations and rate of change of concentrations (i.e. trend analysis) of metals or other materials in oil.

The light, which is fluoresced from, reflected by, absorbed by, or transmitted through the photo-responsive chemicals in the oil is captured in the current design, measured and stored 614. This includes both photo-responsive chemicals that have interacted with metal ions or other material ions in the oil and photo-responsive chemicals that have not interacted with metal or other material ions in the oil. These optical properties may be stored as a function of time as rate and trend data 614. These optical properties and rate and trend data may be converted to concentrations of metals or other materials in the oil, either directly and/or indirectly as concentrations of metal ions or other material ions in the oil. Such concentrations and rate and trend data can then be stored for later use 614.

At this point, some embodiments of the present design have measured and stored value and rate and trend data for concentrations of one or more metals or other materials in oil in a mechanical system. These data may then be compared to previously stored data and/or stored mathematical and/or statistical formulae 616, look-up translation data or other information to determine if an alarm or signal is to be generated 618, including but not limited to one or more of the following signals and/or alarms:
Oil condition has deteriorated;
Oil condition deterioration has accelerated;
Oil change is needed;
Wear in the mechanical system;
Wear in the mechanical system has accelerated;
Contamination has occurred;
Contamination has accelerated; or
Probability of mechanical failure has increased.

These alarms or signals may take one or more forms, including an analog signal or level (including light, frequency, voltage and/or current), digital signal or data, or an indication to one or more of the mechanical system's computers.

If no alarm and/or signal occurs, the current data may be stored with other previously stored data 620. If an alarm or signal occurs, the owner or operator of the mechanical system may receive the alarm or notification 626 and decide whether an oil change is necessary 628. If an oil change is determined necessary, all or part of the previously measured and stored data may be reset 630. If an oil change is necessary and occurs, the sight glass fixture (with one or more photo-responsive chemicals attached) in the mechanical system 632 may be replaced. The process is repeated, starting at 604. Still, if an oil change is necessary and occurs, one or more of the photo-responsive chemicals attached to the sight glass may be rejuvenated by introducing such photo-responsive chemicals to a stronger chelator, which will remove the metal ions or other material ions from the photo-responsive chemicals 632. The process is then repeated, starting at 606. As one non-limiting example, the chemical CN⁻ is a very strong chelator and has been found to chelate metal and other material ions from 8-hydroxyquinoline. However, if an oil change is not necessary, the oil may remain in the mechanical system along with the metal or other materials, metal ions or other material ions, and the photo-responsive chemicals. In this situation, the previously stored data 620 are still relevant and stored, and the process is repeated, starting at 608.

The methods described above may not occur continuously or at a high frequency. These processes can occur once or more every second, minute, hour, or even day depending on the operating parameters of the mechanical system 624 as the result of operating the mechanical system. During this delay period, additional metal or other materials or metal or other material ions may have been introduced to the oil in the mechanical system 624 as the result of operating the mechanical system. The process of reading the changes in the optical properties and correlating these values and trends to machine or oil condition (604-632) is then repeated.

Thus according to the present design, there is provided ion concentration measurement system, comprising an oil mixture comprising an oil potentially containing at least one ion and at least one photo-responsive chemical that changes optical properties in the presence of at least one ion in the oil mixture, photo-detector circuitry configured to receive an optical property from the oil mixture, electronic conversion circuitry configured to convert the optical property into a value representing an ion concentration in the oil mixture, and electronic transmission circuitry configured to transmit a signal or data indicating the value.

According to another aspect of the present design, there is provided a method of measuring concentrations of at least one ion in an oil mixture comprising oil potentially containing at least one ion and at least one photo-responsive chemical that changes at least one optical property in the presence of at least one ion in oil. The method comprises receiving the at least one optical property from the at least one photo-responsive chemical, converting the at least one optical property into a value representing an ion concentration in the oil mixture, and transmitting a signal or data comprising the value.

According to a further embodiment of the present design, there is provided a method of measuring condition of a mechanical system comprising combining data in at least one additional mechanical system capable of measuring concentration of materials in oil with other parameters of the mechanical system, wherein the other parameters comprise first mechanical system operating parameters since last oil change, original oil quality, and first mechanical system current and historic load and operating conditions, implementing statistical learning algorithms configured to learn typical oil or machine degradation patterns for a specific mechanical system type to determine mechanical system-specific oil drain histories, and employing the mechanical system-specific oil drain histories to optimize subsequent oil drain intervals for the first mechanical system.

While the present invention has been particularly shown and described with reference to some embodiments, it will be understood by those skilled in the art that various changes in form and detail may be made therein without departing from the spirit and scope of the invention.

It will be appreciated that variations of the above disclosed and other features and functions, or alternatives thereof, can be desirably combined into many other different systems or applications. Also that various presently unforeseen or unanticipated alternatives, modifications, variations or improvements therein can be subsequently made by those skilled in the art, which are also intended to be encompassed by the present invention.

The foregoing description of specific embodiments reveals the general nature of the disclosure sufficiently that others can, by applying current knowledge, readily modify and/or adapt the system and method for various applications without departing from the general concept. Therefore, such adaptations and modifications are within the meaning and range of equivalents of the disclosed embodiments. The phraseology or terminology employed herein is for the purpose of description and not of limitation.

What is claimed is:

1. An ion concentration measurement system, comprising:
an oil mixture comprising:
an oil potentially containing at least one ion; and
at least one photo-responsive chemical that changes optical properties in the presence of at least one ion in the oil mixture;
photo-detector circuitry configured to receive at least one optical property from the oil mixture;
electronic conversion circuitry configured to convert at least one optical property into a value representing an ion concentration in the oil mixture; and
electronic transmission circuitry configured to transmit a signal or data indicating the value.

2. The ion concentration measurement system of claim 1, further comprising a solid chemical in which the at least one photo-responsive chemical is attached, wherein the solid chemical is attached to one or more mechanical structures.

3. The ion concentration measurement system of claim 1, further comprising an electromagnetic source of at least one wavelength that irradiates upon at least one photo-responsive chemical and induces optical properties in the one photo-responsive chemical.

4. The ion concentration measurement system of claim 1, further comprising a supplemental chemical that increases the rate of conversion of a metal material or non-metal material into ions in the oil mixture.

5. The ion concentration measurement system of claim 1, wherein changes in at least one optical property of at least one photo-responsive chemical converts into a concentration of ions in the oil mixture.

6. The ion concentration measurement system of claim 1, wherein changes in at least one relative optical property of at least one photo-responsive chemical converts into a concentration of ions in the oil mixture, wherein one relative property comprises a difference between one property and a baseline property of at least one photo-responsive chemical.

7. The ion concentration measurement system of claim 1, further comprising a chemiluminescent chemical that reacts with at least one ion or at least one photo-responsive chemical to excite the at least one photo-responsive chemical into a higher energy state, thereby inducing fluorescence.

8. The ion concentration measurement system of claim 1, wherein individual material ion concentrations to be determined are between 100 parts per billion and 1,000 parts per million relative to the oil.

9. The ion concentration measurement system of claim 1, at least one photo-responsive chemical absorbs excitation energy at a wavelength greater than 600 nanometers.

10. The ion concentration measurement system of claim 1, wherein at least one photo-responsive chemical fluoresces in the electromagnetic spectrum at a wavelength greater than 600 nanometers.

11. The ion concentration measurement system of claim 1, wherein the measurement system measures a level of wear in a mechanical system by measuring:
concentration of at least one ion in the oil mixture; or
relative increase in concentration of at least one ion in the oil mixture.

12. The ion concentration measurement system of claim 1, wherein the measurement system measures a level of contamination in a mechanical system by measuring:
concentration of at least one ion in the oil mixture; or
relative increase in concentration of at least one ion in the oil mixture.

13. A method of measuring concentrations of at least one ion in an oil mixture comprising oil potentially containing at least one ion and at least one photo-responsive chemical that changes at least one optical property in the presence of at least one ion in oil, the method comprising;
receiving the at least one optical property from the at least one photo-responsive chemical;
converting the at least one optical property into a value representing an ion concentration in the oil mixture; and
transmitting a signal or data comprising the value.

14. The method of claim 13, wherein at least one photo-responsive chemical attaches to a mechanical structure.

15. The method of claim 13, further comprising illuminating at least one photo-responsive chemical in the oil mixture with at least one wavelength of electromagnetic energy, inducing optical properties in the at least one photo-responsive chemical.

16. The method of claim 13, further comprising exciting at least one photo-responsive chemical into a higher energy state for fluorescence by reacting at least one photo-responsive chemical or at least one ion with a chemiluminescent chemical.

17. The method of claim 13, further comprising increasing conversion rate of at least one metal material or non-metal material in the oil mixture to ions.

18. The method of claim 13, further comprising changing at least one photo-responsive chemical's optical properties based on at least one ion in the oil mixture.

19. The method of claim 13, further comprising changing at least one relative optical properties of at least one photo-responsive chemical based on concentrations of ions in the oil mixture.

20. The method of claim 13, further comprising obtaining measurement of at least one optical property of at least one wavelength emitting from at least one photo-responsive chemical.

21. The method of claim 13, further comprising assessing a level of wear in a mechanical system by measuring a concentration or a relative increase in concentration of at least one ion in the oil mixture.

22. The method of claim 13, further comprising assessing a level of contamination in a mechanical system by measuring a concentration or a relative increase in concentration of at least one ion in the oil mixture.

23. A method of measuring condition of a mechanical system comprising:
combining data in at least one additional mechanical system capable of measuring concentration of materials in oil with other parameters of the mechanical system, wherein the other parameters comprise:
first mechanical system operating parameters since last oil change;
original oil quality; and
first mechanical system current and historic load and operating conditions;
implementing statistical learning algorithms configured to learn typical oil or machine degradation patterns for a specific mechanical system type to determine mechanical system-specific oil drain histories; and
employing the mechanical system-specific oil drain histories to optimize subsequent oil drain intervals for the first mechanical system.
